(19) United States
(12) Patent Application Publication
(10) Pub. No.: US 2020/0021570 A1
(43) Pub. Date: Jan. 16, 2020

(54) BLOCKCHAIN DENTAL IMPLANT SYSTEM

(71) Applicant: Che-Min Lin, Taipei City (TW)

(72) Inventor: Che-Min Lin, Taipei City (TW)

(21) Appl. No.: 16/035,718

(22) Filed: Jul. 16, 2018

(51) Int. Cl. H04L 29/06; G16H 10/60

(52) U.S. Cl. CPC H04L 63/083; H04L 9/0637; G16H 10/60

(57) ABSTRACT

A blockchain dental implant system includes a doctor end and patient end, a doctor at the doctor end generating dental implant medical record data after the diagnosis of a dental implant patient, writing each of the medical records into a connected computer to form a dental implant file, and storing the dental implant file into a web server; the doctor end giving the patient a secret key; the patient end obtaining the secret key, using the key to obtain the dental implant file from a space in connection with the web server and having a right to access to the dental implant file.

### (a) TECHNICAL FIELD OF THE INVENTION

The present invention relates to a blockchain dental implant system, and more particularly to a system using a blockchain form to build up dental implant records.

### (b) DESCRIPTION OF THE PRIOR ART

Conventionally, general patients' consultation data files are recorded through paperwork when they go to see a doctor of a hospital for dental implant, and the doctor will handwrite the diagnosis results, prescription and remarks on the medical record form prepared by the hospital in which personal data is filled after diagnosis. If there are other examinations (such as X-rays) also performed at the same time, they will be placed in the medical record form and filed. When a patient goes back to see the doctor again, the hospital searches the medical record form again according to the patient's data and provides the doctor to complete the new content on it once again, which repeats again and again for future diagnoses. However, it is labor and time consumptive to make, search and file the medical record form. With the progress of software and hardware of information technologies, related applications, for example, electronation of paper medical records, are also developed in a medical field. Some doctors or clinics replace paper medical records with patients' medical record data built up by computer systems for dental implant, improving administrative efficiency and also perfecting medical care.

However, only a hospital computer filing the above computerized medical record data can read it, and patients themselves can even get their medical record only through the cumbersome application procedures of the hospital or even paying fees, causing the patients to be perplexed upon patient transfer or the undertaking of medical records is generated. In some countries, although national health insurance IC card has a medical record in code form, the card itself is not designed to have detailed medical records, causing medical records unable to be fully grasped. Especially, patients themselves have no way to obtain their own medical records.

It can be seen that the circulation of the current medical data is still insufficient. Especially, patients even have less knowledge about their own medical records. Therefore, there are still unresolved problems in the conventional medical records of patients without effective solution at present.

### SUMMARY OF THE INVENTION

One object of the present invention is to provide a blockchain dental implant system, using a blockchain concept to access to a dental implant process.

To achieve the object mentioned above, the present invention proposes a blockchain dental implant system, including a doctor end and patient end, a doctor at the doctor end generating dental implant medical record data after the diagnosis of a dental implant patient, writing each of the medical records into a connected computer to form a dental implant file, and storing the dental implant file into a web server; the doctor end giving the patient a secret key; the patient end obtaining the secret key, using the key to obtain the dental implant file from a space in connection with the web server and having a right to access to the dental implant file.

### BRIEF DESCRIPTION OF THE DRAWINGS

FIG. 1 is a schematically structural block diagram of a preferred embodiment of a blockchain dental implant system according to the present invention.

### DETAILED DESCRIPTION OF THE PREFERRED EMBODIMENTS

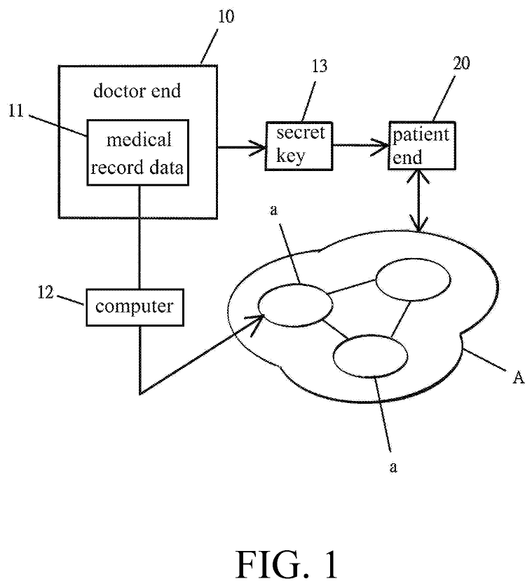

Referring to FIG. 1, a blockchain dental implant system includes a doctor end 10 and patient end 20.

At the doctor end 10, a doctor generates various medical records 11 of dental implant such as time, X-ray films, instruments used, supplementary bone powder, wound healing status, tooth root material model, crown material model, remarks and other messages after the diagnosis of a dental implant patient, and writes each record 11 after the diagnosis of the patient into a connected computer 12 to form a dental implant file and stores it in a block a in connection with a web server, which can be a single one or linked with the adjacent previous blocks a to form a blockchain A, which is a distributed database using cryptographic methods to generate data blocks relatively, and the medical content written in at the doctor end 10 is generated into the data of a data block. Any data in the blockchain A can be obtained from every node in the blockchain A network because blockchain A is mostly open information so that the doctor end 10 must add identification data of the node in relation to the record content in the data block, allowing the dental implant record content in the data block to be obtained only by the patient end 20 obtaining a secret key 13.

The patient end 20 can obtain the secret key 13 from the doctor end 10 and use it to obtain the dental implant file from the blockchain A in connection with the web server. In addition, the patient end 20 has the right to access to the dental implant file.

With the above blockchain dental implant system, the relevant data of the block or dental implant cannot be arbitrarily tampered with, thereby keeping the completeness and safety of the medical records. The blockchain A and its blocks a can be built up on one or more than one web server, and each server is in connection with the other through a network.

The above connection refers to a wireless or wired network signal connection, and the dental implant file any dental implant data of a patient such as diagnosis results, X-ray films, instruments used, supplementary bone powder, wound healing status, tooth root material model, crown material model, remarks and other messages.

The doctor end 10 may be any level of physician who has been trained in dental implant.

The secret key 13 is a set of passwords. Specifically, the secret key 13 has a public key and private key, where the public key is a set of passwords and can be used as a credential to verify the private key after a certificate is issued through authentication, and the private key is a set of passwords, and must be kept by the patient end 20 and further verified with the public key to obtain the dental implant data.

The system structure of the present invention is simple, but the information is stable and safe, the data management and announcement are convenient, and the practical efficiency can be increased effectively.

Especially, patients have the right to access to their own medical records, allowing the application thereof to be simple, convenient and safe.

The present invention has the following advantages over the prior art:
1. the doctor end 10 stores dental implant patients' medical records in the blockchain A safely and completely, providing an innovative service and enhancing the contribution to medical qualities; and
2. patients have the ownership of their own medical records, capable of obtaining their own medical record data directly through network connection and being better for self-health management.

I claim:

1. A blockchain dental implant system, comprising a doctor end and patient end, a doctor at said doctor end generating dental implant medical record data after the diagnosis of a dental implant patient, writing each of said medical records into a connected computer to form a dental implant file, and storing said dental implant file into a web server; said doctor end giving said patient a secret key; said patient end obtaining said secret key, using said key to obtain said dental implant file from a space in connection with said web server and having a right to access to said dental implant file.

2. The system according to claim 1, wherein said doctor end stores said dental implant file into a block in connection with said web server, and said block is linked with adjacent blocks to establish a blockchain.
